(19) United States
(12) Patent Application Publication
(10) Pub. No.: US 2016/0051582 A1
(43) Pub. Date: Feb. 25, 2016

(54) PHARMACEUTICAL COMPOSITION FOR TREATMENT OF CHRONIC PAIN

(71) Applicants: QIFANG LI, Shanghai City (CN); HUA KANG, Shanghai City (CN); The Ninth Hospital Affiliated to Shanghai Jiao Tong University School of Medicine, Shanghai City (CN)

(72) Inventors: QIFANG LI, Shanghai City (CN); HUA KANG, Shanghai City (CN); HONG JIANG, Shanghai City (CN)

(21) Appl. No.: 14/659,608

(22) Filed: Mar. 16, 2015

(30) Foreign Application Priority Data
Aug. 19, 2014 (CN) 201410409247.9

(51) Int. Cl. A61K 33/14; A61K 31/7004; A61K 33/00; A61K 31/167

(52) U.S. Cl. CPC A61K 33/14; A61K 31/167; A61K 31/7004; A61K 33/00

(57) ABSTRACT

A pharmaceutical composition for treatment of chronic pain includes lidocaine, glucose, sodium chloride and sodium bicarbonate. The pharmaceutical composition could be applied for treatment of chronic pains in local position such as soft tissue, muscle tendon, synovial bursa.

### CROSS REFERENCE TO RELATED APPLICATION

The present application is based on and claims priority of Chinese Patent Application No. 201410409247.9, filed on Aug. 19, 2014. The entire disclosure of the above-identified application, including the specification, drawings and claims are incorporated herein by reference in its entirety.

### FIELD OF THE INVENTION

The present invention relates to a pharmaceutical composition, and more particularly to a pharmaceutical composition for treatment of chronic pain.

### BACKGROUND OF THE INVENTION

Various kinds of pains are plaguing millions of patients and chronic pain is the most common and serious one. The idea that “Chronic pain is a disease” has basically been accepted by social and medical community, it causes a series of pathophysiological changes in and around central nervous system, and reflects different pathological changes through specific symptoms and signs. As well as multiple and persistent, chronic pain has seriously affected patient's work and life.

In order to reduce patient's pain, clinicians has consecutively studied for effective means of treating chronic pain, including oral drug therapy, the principal oral drugs including non-steroidal anti-inflammatory painkillers, cyclooxygenase (COX) inhibitors, receptor agonist baclofen (pregabalin), and sodium channel antagonists (carbamazepine controlled release agent); TCM (Traditional Chinese Medicine) therapies, including massage, acupuncture, cupping, or etc; physical therapies, including ultra-laser, low-frequency impulse electrotherapy; invasive therapies, including pain point injections, nerve blocks, spinal blocks, RF, small needle knife therapy, and surgeries such as minimally invasive surgery; and local injection therapies, the principal injection drugs including non-steroidal anti-inflammatory painkillers and glucocorticoids.

Local injection therapy is an effective and widely used method in treating chronic pain. The local injection drug glucocorticoids have a good therapeutic effect on aseptic inflammation of chronic pain, but the application of hormones has long been controversial. Furthermore, glucocorticoids only improve the chronic pain in a short term but without a significant long-term effect. In addition, repeated and large-scale application of glucocorticoids may also lead to high blood pressure, high blood sugar, peptic ulcer disease, infectious disease, corneal ulcers, articular cartilage microcirculation disturbance, or influence articular cartilage metabolism and accelerate the degeneration of articular cartilage. Especially for some special patients such as high blood pressure, diabetes, and immunocompromised patients, the application of glucocorticoids are limited. That is to say, the application of hormone drugs has shortcomings and limitations.

### SUMMARY OF THE INVENTION

The present invention provides a pharmaceutical composition which could be used in most chronic pain patients.

The applicant has found that a pharmaceutical composition including sodium bicarbonate, lidocaine, glucose and sodium chloride has a significant effect on most chronic pain sites such as soft tissue, muscle tendon, synovial bursa.

The pharmaceutical composition includes lidocaine, glucose, sodium chloride, and sodium bicarbonate.

In a preferred embodiment of the present invention, the pharmaceutical composition is a medical solution.

In a preferred embodiment of the present invention, the pharmaceutical composition includes 0.1% to 0.3% by mass to volume ratio of lidocaine, 1.25% to 2.5% by mass to volume ratio of glucose, 0.045% to 0.135% by mass to volume ratio of sodium chloride and 3% to 3.75% by mass to volume ratio of sodium bicarbonate and water.

In a preferred embodiment of the present invention, the pharmaceutical composition is formulated by 1% to 4% lidocaine solution, 10% to 50% glucose solution, 2% to 10% sodium bicarbonate solution and 0.9% sodium chloride solution.

In a preferred embodiment of the present invention, 20 ml of the pharmaceutical composition includes 1 to 3 ml of 2% lidocaine solution, 1 to 2 ml of 25% glucose solution, 1 to 3 ml of 0.9% sodium chloride solution, and 12 to 15 ml of 5% sodium bicarbonate solution.

In a preferred embodiment of the present invention, 20 ml of the pharmaceutical composition comprises 1 ml of 2% lidocaine solution, 1 ml of 25% glucose solution, 1 ml of 0.9% sodium chloride solution, and 12 to 15 ml of 5% sodium bicarbonate solution.

In a preferred embodiment of the present invention, the pharmaceutical composition is used for local injection.

In preferred embodiments of the present invention, the pharmaceutical composition could be used in treatment of most chronic pain sites such as soft tissue, muscle tendon, synovial bursa.

In preferred embodiments of the present invention, the pharmaceutical composition is used for treatment of frozen shoulder, knee bursitis and the third lumbar vertebral transverse process syndrome.

In the embodiment that the pharmaceutical composition is used for treatment of frozen shoulder, 20 ml of the pharmaceutical composition includes 1 ml of 2% lidocaine solution, 1 ml of 25% glucose, 3 ml of 0.9% sodium chloride solution and 15 ml of 5% sodium bicarbonate solution. The pharmaceutical composition is locally injected in soft tissue around shoulder joint and/or rotator cuff in a dose of 5-15 ml in each pain site with the total dose for each patient in the range from 10-80 ml.

In the embodiment that the pharmaceutical composition is used for treatment of knee bursitis, 20 ml of the pharmaceutical composition includes 1.5 ml of 2% lidocaine solution, 2 ml of 25% glucose, 2 ml of 0.9% sodium chloride solution and 14.5 ml of 5% sodium bicarbonate solution. The pharmaceutical composition is locally injected in knee bursa and/or soft tissue therearound in a dose of 5-15 ml in each pain site with the total dose for each patient in the range from 10-40 ml.

In the embodiment that the pharmaceutical composition is used for treatment of the third lumbar vertebral transverse process syndrome, 20 ml of the pharmaceutical composition includes 1 ml of 2% lidocaine solution, 2 ml of 25% glucose, 3 ml of 0.9% sodium chloride solution and 14 ml of 5% sodium bicarbonate solution. The pharmaceutical composition is fully infiltration injected at the tip of the third lumbar vertebral transverse process, along and around the transverse tip and its upper and lower edges, in a dose of 10-15 ml in each pain site with the total dose for each patient in the range from 15-45 ml. The pharmaceutical composition is injected weekly or biweekly and the total injection times are in the range from 3-5 times per course of treatment.

### BRIEF DESCRIPTION OF THE DRAWINGS

No drawings

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

Sodium bicarbonate (NaHCO₃) is an alkaline agent capable of reducing irritation caused by lactic acid and its metabolites in partial sites of chronic pain and indirectly relieving muscle spasm; while alkalizing partial sites of the chronic pain and restraining generation and development of sterile inflammation. Sodium bicarbonate is also capable of constricting diastolic state blood vessels, dilating spastic state blood vessels, maintaining these vessels' normal vasomotor function, improving nutrients and oxygen supply of local tissue; causing fibrin decomposition within proliferative connective tissue, absorbing and dissipating local cable strips, and relieving the peripheral nerve compression and pain.

Lidocaine hydrochloride is an amide local anesthetic with pain blocking effect, and capable of easing local muscle spasm, greatly improving tissue circulation, promoting tissue metabolism and increasing tissue oxygen supply, reducing inflammatory edema and accelerating tissue repair.

Glucose is body's necessary energy and nutrients for metabolism. Aseptic inflammation of chronic pain sites can lead to localized metabolic disorders and nutritional malabsorption of chronic pain sites, however, glucose can provide high-energy nutrients to local sites of chronic pain, improve energy and nutrient supply of local pain sites, promote body repair, alleviate and eliminate chronic pain.

Sodium chloride (NaCl) is an indispensable inorganic salt for human body. 0.9% sodium chloride solution is also known as physiological saline, which is capable of maintaining osmotic pressure and acid-base balance inside and outside cells of local pain sites, and preventing dehydration. In the present invention, 0.9% sodium chloride solution represents that there is 0.9 g sodium chloride in 100 ml sodium chloride solution.

The pharmaceutical composition of the present invention includes 0.1% to 0.3% by mass to volume ratio of lidocaine, 1.25% to 2.5% by mass to volume ratio of glucose, 0.045% to 0.135% by mass to volume ratio of sodium chloride, 3% to 3.75% by mass to volume ratio of sodium bicarbonate, and appropriate volume of water. In the present embodiment, % represents the ingredient concentration in the pharmaceutical composition. For example, 0.1% to 0.3% of lidocaine represents there is 0.1 to 0.3 g lidocaine in 100 ml pharmaceutical composition.

It should be noted that the lidocaine, glucose, sodium chloride and sodium bicarbonate in the present pharmaceutical composition are medical and pharmaceutical preparations.

Because the content of ingredients in the pharmaceutical composition are low and the pharmaceutical composition is usually used by means of local injection, the pharmaceutical composition is usually prepared by medical solutions of special concentrations, therefore, relative content errors of the ingredients can be reduced, and the prepared pharmaceutical compositions could be injected in the patient directly.

The medical solutions used for preparing the pharmaceutical composition can be finished medical solution prepared by pharmaceutical companies or hospitals, and the finished medical solution may have a variety of concentrations.

The lidocaine solution used for preparing the pharmaceutical composition may be 1% to 4% lidocaine solution, including 1%, 2% and 4% lidocaine solutions; the glucose solution used for preparing the pharmaceutical composition may be 10% to 50% glucose solution, including 10%, 20%, 25% and 50% glucose solutions; the sodium bicarbonate solution used for preparing the pharmaceutical composition may be 2% to 10% bicarbonate solution, including 2%, 5% and 10% bicarbonate solutions; and the sodium chloride solution used for preparing the pharmaceutical composition is 0.9% sodium chloride solution. The 0.9% sodium chloride solution is a common medical solution which is known as physiological saline. In the present embodiment, % represents the ingredient concentration of the solution. For example, 1% lidocaine solution represents there is 1 g lidocaine in 100 ml lidocaine solution.

Preferably, the pharmaceutical composition solution includes 1 to 3 ml of 2% lidocaine solution, 1 to 2 ml of 25% glucose solution, 1 to 3 ml of 0.9% sodium chloride solution, 12 to 15 ml of 5% sodium bicarbonate solution and appropriate volume of water. This results in a total volume of 20 ml of pharmaceutical composition solution (containing 0.02 to 0.06 g of lidocaine, 0.25 to 0.5 g of glucose, 0.009 to 0.027 g of sodium chloride, 0.6 to 0.75 g of sodium bicarbonate). The volume of the lidocaine solution, glucose solution, sodium chloride solution, and sodium bicarbonate solution can be changed according to the total volume of the pharmaceutical composition solution.

More concretely, the pharmaceutical composition solution includes 1 ml of 2% lidocaine solution, 1 ml of 25% glucose solution, 1 ml of 0.9% sodium chloride solution, 12 to 15 ml of 5% sodium bicarbonate solution and appropriate volume of water. This results in a total volume of 20 ml of pharmaceutical composition solution. The volume of the lidocaine solution, glucose solution, sodium chloride solution, and sodium bicarbonate solution can be changed according to the total volume of the pharmaceutical composition solution.

Because there are no chemical reactions between sodium bicarbonate, lidocaine, glucose and sodium chloride under room temperature, the obtained pharmaceutical composition could be more stable and non-toxic after use in patients, the compatible pharmaceutical compositions could be stored in sterile conditions for a long period of time.

The pharmaceutical compositions could be used for treating chronic pains in soft tissue, muscle tendon, or synovial bursa, by eliminating nerve block and other symptoms through localized injection, and the chronic pain is eliminated accordingly.

Hereinafter, some embodiments of the applications of the pharmaceutical composition are shown in details.

### Embodiment 1

### The Pharmaceutical Composition Being Applied for Treatment of Frozen Shoulder

Frozen shoulder is a kind of aseptic inflammation generated around soft tissue of shoulder joint, with joint pain and dysfunction as main symptoms. Common treatment methods of shoulder joint include oral medications, physical therapies such as massage, functional exercises or etc. Localized injection of hormones such as corticosteroids will be used for treatment if results of the oral medications and physical therapies are not satisfied.

The pharmaceutical composition for treatment of frozen shoulder is formulated in a sterile environment, and the method for formulating such pharmaceutical composition includes: respectively drawing 1 ml of 2% lidocaine solution, 1 ml of 25% glucose, 3 ml of 0.9% sodium chloride solution and 15 ml of 5% sodium bicarbonate solution into a sterile syringe; mixing the solutions together to formulate 20 ml of compatibility pharmaceutical composition in a sterile pharmaceutical container. The formulated pharmaceutical composition is drawn into a sterile syringe waiting for use. In other embodiments, the volumes of the solutions and the pharmaceutical composition solution could be changed according to the actual requirement of injection volume.

Patients: We studied clinical effects of the pharmaceutical composition in patients aged from 25 to 80 years, no matter male or female, with a history of frozen shoulder from about 1 month to 3 years.

Directions: The pharmaceutical composition is locally injected in pain sites such as soft tissue around shoulder joint, rotator cuff, and/or etc.

Dose: 5-15 ml of the pharmaceutical composition solution is injected in each pain site of the frozen shoulder patient. Multiple area injection is suitable for patients with multiple pain sites and the total dose for each patient is controlled in the range from 10-80 ml.

Injection frequencies: The patient is injected weekly or biweekly. The injection times can be regulated according to severity of the patient's condition, and the total injection times is controlled in the range from 3-5 times per course of treatment.

Clinical effects: The patient generally feels the injection site slightly swelling at the injection day. The chronic pain at the primary pain site gradually eases three days later. Generally speaking, chronic pains of mild patients could be relieved after one injection; chronic pains of ordinary patients could be substantially relieved after 1-2 times injection; chronic pains of intractable, wide-range painful patients could be relieved after 3-5 courses of injection. Because of the individual differences of patients and pain cases, the statistical results are based on the actual condition of the clinical patients.

### Embodiment 2

### The Pharmaceutical Composition Being Applied for Treatment of Knee Bursitis

In clinical practice, knee bursitis is one of the most common reasons causing knee pains. Knee bursa is vulnerable to injure because of frequent knee joint movement. The knee bursa injury includes acute trauma and repetitive micro trauma. Common knee bursa injury includes suprapatellar bursitis, prepatellar bursitis, infrapatellar bursitis, anserine bursitis, or etc.

The pharmaceutical composition for treatment of the knee bursitis is formulated in a sterile environment, and the method for formulating such pharmaceutical composition includes: respectively drawing 1.5 ml of 2% lidocaine solution, 2 ml of 25% glucose, 2 ml of 0.9% sodium chloride solution and 14.5 ml of 5% sodium bicarbonate solution into a sterile syringe; mixing the solutions together to formulate 20 ml of compatibility pharmaceutical composition in a sterile pharmaceutical container. The formulated pharmaceutical composition is drawn into a sterile syringe waiting for use. In other embodiments, the volumes of the solutions and the pharmaceutical composition solution could be changed according to the actual requirement of injection volume.

Patients: We studied clinical effects of the pharmaceutical composition in patients aged from 25 to 80 years, no matter male or female, with a history of knee bursitis about 1 month to 3 years.

Directions: The pharmaceutical composition is locally injected in pain sites such as knee bursa and/or soft tissue therearound.

Dose: 5-15 ml of the pharmaceutical composition solution is injected in each pain site of the knee bursitis patients. Multiple area injection is suitable for patients with multiple pain sites and the total dose for each patient is controlled in the range from 10-40 ml.

Injection frequencies: The patient is injected weekly or biweekly. The injection times can be regulated according to severity of the patient's condition, and the total injection times is controlled in the range from 3-5 times per course of treatment.

Clinical effects: the patient generally feels the injection site slightly swelling at the injection day. Strenuous exercise is forbidden while moderate exercise is allowed after injection. Generally speaking, chronic pains of mild patients could be relieved after one injection; chronic pains of ordinary patients could be substantially relieved after 1-2 times injection; chronic pains of intractable patients could be relieved after 3-5 courses of injection. Because of the individual differences of patients and pain cases, the statistical results are based on the actual condition of the clinical patients.

### Embodiment 3

### The Pharmaceutical Composition Being Applied for treatment of the third lumbar vertebral transverse process syndrome

The third lumbar vertebral transverse process syndrome is a common disease of lumbar pain or lumbar-leg pain patients, which usually onsets in young manual laborer. Because the third lumbar vertebral transverse process is particularly long and extends horizontally, with blood vessels and nerve bundles extending nearthrough and attached more muscular fascia, the third lumbar vertebral locates at the vertex of the lumbar lordosis curvature and at an important position of bearing mechanical transmission, therefore, the third lumbar vertebral transverse process is susceptible to external force, and the attached muscle is susceptible to tear, bleeding, scar adhesions, thickened fascia contracture, so that the neurovascular bundle is susceptible to generate pain symptoms because of friction, irritation and compression.

The pharmaceutical composition for treatment of the third lumbar vertebral transverse process syndrome is formulated in a sterile environment, and the method for formulating such pharmaceutical composition includes: respectively drawing 1 ml of 2% lidocaine solution, 2 ml of 25% glucose, 3 ml of 0.9% sodium chloride solution and 14 ml of 5% sodium bicarbonate solution into a sterile syringe; mixing the solutions together to formulate 20 ml of compatibility pharmaceutical composition in a sterile pharmaceutical container. The formulated pharmaceutical composition is drawn into a sterile syringe waiting for use. In other embodiments, the volumes of the solutions and the pharmaceutical composition solution could be changed according to the actual requirement of injection volume.

Patients: We studied clinical effects of the pharmaceutical composition in patients aged from 25 to 70 years, no matter male or female, with a history of the third lumbar vertebral transverse process syndrome about 1 month to 3 years.

Directions: the needle is thrust in a depth of 3 to 5 cm at the third lumbar vertebral transverse process, along a direction of 45 degrees, until it touches the tip of the third lumbar vertebral transverse process, in case the needle is withdrawn without blood, full infiltrating injection could be performed along and around the transverse tip and its upper and lower edges. Likewise, if another transverse tip needs injection, the needle can be pulled out till underneath skin and change the direction to reach the another transverse tip for injection.

Dose: 10-15 ml of the pharmaceutical composition solution is injected in each pain site of the third lumbar vertebral transverse process syndrome patients. Multiple area injection is suitable for patients with multiple pain sites and the total dose for each patient is controlled in the range from 15-45 ml.

Injection frequencies: The patient is injected weekly or biweekly. The injection times can be regulated according to severity of the patient's condition, and the total injection times is controlled in the range from 3-5 times per course of treatment.

Clinical effects: The patient generally feels the injection site slightly swelling at the injection day. It is better to have a rest after injection. Generally speaking, chronic pains of mild patients could be relieved after one injection; chronic pains of ordinary patients could be substantially relieved after 1-2 times injection; and chronic pains of intractable patients could be relieved after 3-5 courses of injection. Because of the individual differences of patients and pain cases, the statistical results are based on the actual condition of the clinical patients.

### CONCLUSION

From the clinical effects of the forgoing embodiments, we can conclude that the pharmaceutical composition of the present invention is suitable for effectively treating the chronic pains of local positions such as soft tissue, muscle tendon, synovial bursa.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A pharmaceutical composition for treatment of pain comprising: lidocaine, glucose, sodium chloride and sodium bicarbonate:
wherein the pharmaceutical composition comprises 0.1% to 0.3% by mass to volume ratio of lidocaine, 1.25% to 2.5% by mass to volume ratio of glucose, 0.045% to 0.135% by mass to volume ratio of sodium chloride and 3% to 3.75% by mass to volume ratio of sodium bicarbonate and water.

2. The pharmaceutical composition according to claim 1, wherein the pharmaceutical composition is medical solution.

3. (canceled)

4. The pharmaceutical composition according to claim 1, wherein the pharmaceutical composition is formulated by 1% to 4% lidocaine solution, 10% to 50% glucose solution, 2% to 10% sodium bicarbonate solution and 0.9% sodium chloride solution.

5. The pharmaceutical composition according to claim 4, wherein 20 ml of the pharmaceutical composition comprises 1 to 3 ml of 2% lidocaine solution, 1 to 2 ml of 25% glucose solution, 1 to 3 ml of 0.9% sodium chloride solution, and 12 to 15 ml of 5% sodium bicarbonate solution.

6. The pharmaceutical composition according to claim 5, wherein 20 ml of the pharmaceutical composition comprises 1 ml of 2% lidocaine solution, 1 ml of 25% glucose solution, 1 ml of 0.9% sodium chloride solution, and 12 to 15 ml of 5% sodium bicarbonate solution.

7. The pharmaceutical composition according to claim 1, wherein the pharmaceutical composition is used for local injection.

8. The pharmaceutical composition according to claim 1, wherein the pain to be treated comprises chronic pain which comprises soft tissue pain, muscle tendon pain and synovial bursa pain.

9. A pharmaceutical composition for treatment of pain comprising: lidocaine, glucose, sodium chloride and sodium bicarbonate:
wherein 20 ml of the pharmaceutical composition comprises 1 to 3 ml of 2% lidocaine solution, 1 to 2 ml of 25% glucose solution, 1 to 3 ml of 0.9% sodium chloride solution, and 12 to 15 ml of 5% sodium bicarbonate solution.

10. The pharmaceutical composition according to claim 9, wherein the pharmaceutical composition is used for treatment of frozen shoulder, and 20 ml of the pharmaceutical composition for treatment of frozen shoulder comprises 1 ml of 2% lidocaine solution, 1 ml of 25% glucose, 3 ml of 0.9% sodium chloride solution and 15 ml of 5% sodium bicarbonate solution.

11. The pharmaceutical composition according to claim 9, wherein the pharmaceutical composition is used for treatment of frozen shoulder, and is locally injected in soft tissue around shoulder joint and/or rotator cuff.

12. The pharmaceutical composition according to claim 9, wherein the pharmaceutical composition is used for treatment of frozen shoulder, and is injected 5-15 ml in each pain site of the frozen shoulder patient and the total dose for each patient is in the range from 10-80 ml.

13. The pharmaceutical composition according to claim 9, wherein the pharmaceutical composition is used for treatment of knee bursitis, and 20 ml of the pharmaceutical composition comprises 1.5 ml of 2% lidocaine solution, 2 ml of 25% glucose, 2 ml of 0.9% sodium chloride solution and 14.5 ml of 5% sodium bicarbonate solution.

14. The pharmaceutical composition according to claim 9, wherein the pharmaceutical composition is used for treatment of knee bursitis, and is locally injected in knee bursa and/or soft tissue therearound.

15. The pharmaceutical composition according to claim 9, wherein the pharmaceutical composition is used for treatment of knee bursitis, and is injected 5-15 ml in each pain site of the knee bursitis patient and the total dose for each patient is in the range from 10-40 ml.

16. A pharmaceutical composition for treatment of the third lumbar vertebral transverse process syndrome comprising: lidocaine, glucose, sodium chloride and sodium bicarbonate:
wherein 20 ml of the pharmaceutical composition comprises 1 ml of 2% lidocaine solution, 2 ml of 25% glucose, 3 ml of 0.9% sodium chloride solution and 14 ml of 5% sodium bicarbonate solution.

17. (canceled)

18. The pharmaceutical composition according to claim 16, wherein the pharmaceutical composition is fully infiltration injected at the tip of the third lumbar vertebral transverse process, along and around the transverse tip and its upper and lower edges.

19. The pharmaceutical composition according to claim 16, wherein the pharmaceutical composition is injected weekly or biweekly and the total injection times is in the range from 3-5 times per course of treatment.

20. The pharmaceutical composition according to claim 16, wherein the pharmaceutical composition is injected 10-15 ml in each pain site of the third lumbar vertebral transverse process syndrome patient and the total dose for each patient is in the range from 15-45 ml.
